(19) United States
(12) Patent Application Publication
(10) Pub. No.: US 2009/0041860 A1
(43) Pub. Date: Feb. 12, 2009

(54) ORALLY-DISPERSIBLE PHARMACEUTICAL COMPOSITIONS

(75) Inventors: Alvaro Acebron Fernandez, Madrid (ES); Dolores Blanco Lousame, Madrid (ES); Candida Gasco Guerrero, Madrid (ES)

Correspondence Address: SHERIDAN ROSS PC, 1560 BROADWAY, SUITE 1200, DENVER, CO, 80202, US

(73) Assignee: ITALFARMACO, S.A., Alcobendas (ES)

(21) Appl. No.: 11/577,861

(22) PCT Filed: Oct. 24, 2005
(86) PCT No.: PCT/ES05/00566

§ 371 Date: Jan. 28, 2008

(30) Foreign Application Priority Data
Oct. 25, 2004 (ES) P200402560

(51) Int. Cl. A61K 33/16; A61K 33/06; A61K 33/10; A61P 19/10

(52) U.S. Cl. 424/673; 424/682; 424/687

(57) ABSTRACT

The invention relates to oral pharmaceutical compositions which take the form of orally-dispersible tablets containing calcium and, optionally, vitamin D and/or fluorine together with pharmaceutically-acceptable excipients and which can be used for the treatment or prevention of osteoporosis and other diseases characterised by loss of bone mass.

The present invention concerns oral pharmaceutical compositions in the form of orally dispersible tablets that contain calcium and optionally vitamin D and/or fluorine combined with pharmaceutically acceptable excipients useful for treatment or prevention of osteoporosis and other diseases characterized by loss of bone mass.

### STATE OF THE ART

The skeleton performs functions in support and protection of the soft organs as well as in mineral homeostasis and acid base homeostasis. To perform such functions bone possesses a complex anatomical-functional organization.

In bone composition we distinguish a solid organic matrix (osteoid) formed by type I collagen, strengthened with a deposit of mineral salts of calcium, magnesium, phosphate, carbonate, citrate, chlorine and fluorine. The most abundant salt is hydroxyapatite (Ca₁₀(PO₄)₆(OH)₂), which forms crystals interwoven with the collagen fibers.

In the bone of a normal adult the collagen fibers are preferentially arranged in layers, forming a lamellar structure. The bone lamellae have cavities that house cells (osteoclasts, osteoblasts and osteocytes) and are connected by Haversian canals through which interstitial liquids and blood circulate.

With respect to a structural organization two types of bone are recognized: cortical, compact and with thick layers of calcified osteoid, and trabecular, less dense and with numerous bone spikes.

Bone formation begins in the uterus and continues during infancy and adolescence to skeletal maturity. Over the remaining life, the bone is not a static element but is a very active tissue that is constantly renewed through a mechanism of remodeling. The mechanism consists of destruction of small amounts of bone carried out by osteoclasts (bone resorption) followed by their replacement by osteoblasts (bone formation). This activity is regulated both by systemic factors (parathyroid hormone, calcitonin, vitamin D) and local factors (cytokines, growth factors and peptides) and is affected by drugs, habits and various pathologies.

Osteoporosis is the most common alteration of the skeleton in which a reduction of bone mass is produced per unit volume with respect to what is considered normal for a specific age, sex and race, but the relation between organic and mineral phases is maintained. This relation can be altered in other metabolic bone diseases, like osteomalacia.

Osteoporosis can be of distinct types: primary, secondary, idiopathic and localized. Primary osteoporosis has as causal factors menopause (type I, characterized by loss of trabecular bone) and aging (type II, characterized by loss of cortical bone). Secondary osteoporosis can be caused by different diseases, certain drugs or prolonged immobilization. Idiopathic osteoporosis has an unknown cause.

Under normal conditions, bone loss produced by osteoclastic activity is restored by osteoblastic activity. However, in idiopathic osteoporosis, an imbalance between formation and resorption occurs either from an excess of activity of osteoclasts or a reduction in activity of osteoblasts. In type I osteoporosis, osteoclastic activity predominates and loss of bone mass is greater than under normal conditions, osteoblastic activity is normal but is not capable of compensating for the loss of bone mass. In type II osteoporosis, osteoclastic activity is normal but osteoblastic activity is reduced and is not capable of compensating for the loss.

Calcium is the predominant mineral in bone and normally is supplied to the body through the diet. However, in certain cases this supply is not sufficient and supplements of this mineral must be prescribed in order to prevent or treat osteoporosis and other diseases characterized by loss of bone mass.

The amount of recommended calcium varies as a function of the authors, age of the person and the administered salt. Generally the recommended amount for an adult is between 800 and 1500 mg/day.

The term vitamin D refers to a group of molecules with a steroid structure, including cholecalciferol (vitamin D3), ergocalciferol (vitamin D2), its biologically active metabolites (especially 1,25-dihydroxycholecalciferol) and its precursors (provitamin D2 present in foods of plant origin, provitamin D3 available in foods of animal origin).

This vitamin promotes intestinal absorption of calcium phosphate and magnesium ions, contributes to regulation of calcium in the plasma and by acting in the process of formation and resorption of bone and stimulates resorption of calcium in the kidney. Overall the crucial effect of vitamin D on bone is to provide an appropriate balance of calcium and phosphorus to contribute to mineralization.

Vitamin D is produced in the body by the action of sun rays on precursors obtained from foods. However, in situations of deficient diet and/or rare exposure to sunlight, it will be necessary to administer supplements with vitamin D to prevent or treat osteoporosis and other bone diseases.

All the forms of vitamin D are liposoluble and accumulate in the body. They should not be administered in supraphysiological concentrations because they alter renal, neurological and digestive function. The recommended amounts of vitamin D vary between 200 and 800 IU/day, depending on the age and nutritional situation of the person.

Fluorine is mostly associated with calcified tissues (bones and teeth). It is a known inductor of osteoblastic proliferation. Normally it is supplied to the body through water and foods but in some cases it can be necessary to prescribe supplements, which are sodium fluoride (NaF) and sodium monofluorophosphate (MFP), the only clinically acceptable salts.

The optimum therapeutic range of fluorine ions is from 10 to 50 mg/day for an adult human. It is recommended that no more than 30 mg fluorine/day be administered, since it has been observed that when doses of 60 mg NaF/day are used, formation of abnormal bone is produced.

The use of compositions that contain calcium and optionally vitamin D and/or fluorine in the prevention of treatment of osteoporosis is widely known.

In patent application WO 99/06051 pharmaceutical compositions containing calcium in the form of a salt, preferably calcium phosphate, vitamin D and a binder chosen from propylene glycol, polyethylene glycol with a molecular weight between 300 and 1500, liquid paraffin or silicone oil are described.

In document WO 96/09036 therapeutic combinations of vitamin D and a calcium salt, preferably calcium carbonate are described, which also contain a first binder in synergistic combination with a diluent, second binder and a lubricant, which is a diluent and the second binder is a sweetener. The first binder is chosen from cellulose, maltodextrin and polyvinylpyrrolidone, the diluent is preferably xylitol, the second binder in the preferred form is sorbitol and the lubricant is generally magnesium stearate.

Nutritional substances containing calcium, specifically in the form of calcium malate-citrate salt, vitamin D and optionally an estrogen are described in document WO 92/19251.

Multilayer oral tablets with a central core of calcium salt and an external coating of fluoride, useful as a nutritional substance during pregnancy are described in document U.S. Pat. No. 3,345,265.

However, the pharmaceutical formulations of calcium commercially available now have a number of problems that do not make them acceptable for everyone.

Tablets and capsules are the preferred pharmaceutical forms for administration of drugs because they can be precisely dosed, are easily produced on a large scale and contribute to good compliance with treatment.

However, some patients have difficulty in taking tablets and hard gelatin capsules and, as a result, do not take the medication as prescribed.

Orally dispersible tablets can be defined as solid forms that disintegrate or dissolve instantaneously or rapidly in the oral cavity without a need to administer liquids that form a solution or suspension that can be taken easily. They are also called fast melt tablets, quick dissolving tablets, orodispersible, bucodisintegrable, orodisintegrable or bucosoluble tablets.

These pharmaceutical forms are very useful in persons, who suffer from dysphagia and have difficulty in swallowing tablets or traditional capsules, a fact that is particularly acute in the elderly and children. They are also useful in persons, who do not have easy access to water, for example, vehicle drivers, attendees at long meetings, etc.

Orally dispersible tablets can be prepared by different methods, mostly freeze drying (lyophilization), formation by molding and direct compression.

The first two methods permit preparation of orally dispersible tablets that disintegrate in about 30 seconds but have low physical strength and high friability. On the other hand, direct compression provides tablets with greater friability but which disintegrate over a longer time.

### SUMMARY OF THE INVENTION

There is consequently a need to have tablets which:

permit effective dosing and are nontoxic in terms of calcium and optionally vitamin D and/or fluorine, for treatment of osteoporosis and other diseases related to loss of bone mass,

undergo rapid disintegration in contact with saliva and good palatability, improving the convenience and compliance by the patient at the same time as efficacy of treatment,

exhibit pharmacotechnical characteristics such as adequate fluidity and easy compression of their components along with friability and adequate hardness of the final functional form,

have a competitive cost and comparative advantages with other pharmaceutical forms.

In the first place, it must be kept in mind that it is preferable to have tablets with a content of calcium salt between 45 and 90% by weight of the formulation in order to achieve the recommended daily ingestion of calcium administered in one or two tablets per day. But this active agent in increased percentage causes disagreeable palatability and limits the amount of excipients that can be incorporated in the formulation. The less acceptable taste and texture are therefore a problem that is difficult to solve in this type of formulation.

The inventors of the present invention surprisingly found that the combination of large amounts of calcium salts with excipients with specific characteristics permits orally dispersible tablets to be obtained with an increased percentage of calcium salt and acceptable organoleptic properties.

On the other hand, it must be kept in mind in general that the effectiveness of disintegration is strongly affected by the size and hardness of the orally dispersible tablet. Optimal disintegration properties are often associated with medium or small sizes and/or low physical strength (high friability and low hardness).

As a result, a difficulty inherent to formulations in the form of orally dispersible tablets is to obtain adequate friability and hardness values as well as acceptable disintegration times.

According to European Pharmacopoeia 4.1, 2002 the disintegration time of an orally dispersible tablet must be less than 3 minutes.

The inventors of the present invention surprisingly found that, despite the limitation represented by the amount of excipients that can be incorporated in the composition, the use of certain excipients permits orally dispersible tablets to be obtained of a calcium salt and optionally vitamin D and/or fluorine salt by a direct compression method which satisfy the disintegration time required by the pharmacopoeia and at the same time have an adequate size, integrity and mechanical strength that permits them to be transported without fragility. At the same time orally dispersible tablets with adequate friability and hardness can be obtained.

To summarize, the excipients used in the present invention permit tablets with rapid disintegration in the mouth and at the same time acceptable taste and texture, which provides convenience to the patient and as a result improves compliance with treatment.

In addition, the orally dispersible tablets of the present invention, being obtained by direct compression, have a lower manufacturing cost than, for example, lyophilized tablets, since they use conventional production and packaging equipment, commonly available excipients in a limited number of steps.

At the same time, they enjoy clear advantages with respect to other pharmaceutical forms. In comparison with liquid forms, they permit administration of an exact dose of active principle and have better chemical stability and microbiological properties. Compared with chewable oral forms, they dissolve instantaneously in the oral cavity without requiring administration of liquids or chewing. In comparison with effervescent tablets, they have fewer production and preservation difficulties and at the same time do not require preparation prior to administration (they do not have to be dispersed beforehand in water) so that they enjoy better acceptance by the patient.

Additionally, the orally dispersible tablets permit the active principles to be immediately available to be absorbed and can do so before reaching the stomach through the oral mucosa, pharyngeal mucosa and esophagus.

As a result, the first aspect of the present invention pertains to oral pharmaceutical compositions in the form of orally dispersible tablets, which contain as active principle(s) elemental calcium and optionally vitamin D and/or fluorine combined with pharmaceutically acceptable excipients.

The second aspect of the present invention pertains to use of the orally dispersible tablets of calcium and optionally vitamin D and/or fluorine in the prevention or treatment of osteoporosis and other diseases characterized by loss of bone mass.

A third aspect of the present invention pertains to a method for preparation of the orally dispersible tablets of calcium and optionally vitamin D and/or fluorine by direct compression.

### DETAILED DESCRIPTION

The formulations of this invention preferably contain elemental calcium in an amount between 15 and 35 wt % of the formulation, at least one disintegrant and at least one sweetener.

In a preferred variant the orally dispersible tablets of the present invention contain:
between 400 and 700 mg elemental calcium;
at least one disintegrant;
at least one sweetener.

In another preferred variant the orally dispersible tablets of the present invention contain:
between 400 and 700 mg elemental calcium;
between 100 and 500 IU vitamin D;
at least one disintegrant;
at least one sweetener.

In another preferred variant the orally dispersible tablets of the present invention contain:
between 400 and 700 mg elemental calcium;
between 100 and 500 IU vitamin D;
between 2 and 30 mg fluorine;
at least one disintegrant;
at least one sweetener.

The elemental calcium is supplied in the form of a salt.

Preferably the calcium salt is chosen among calcium carbonate, calcium pidolate, calcium lactate, calcium citrate, calcium gluconate, calcium chloride, calcium glucoheptonate, calcium glycerophosphate, calcium phosphate and their mixtures.

In a more preferred variant the calcium salt is chosen from the group formed by calcium carbonate, calcium gluconate, calcium lactate, calcium citrate and their mixtures.

In an even more preferred variant the calcium salt is calcium carbonate in a percentage between 45 and 90% by weight of the formulation, more preferably between 70 and 80%. In particular, tablets with a weight between 2000 and 2200 mg and with a content of calcium carbonate between 1000 and 1750 mg are preferred, more preferably between 1250 and 1750 mg. This salt has a double function as source of calcium and as an agent capable of releasing carbon dioxide (CO₂) in the presence of an acid at the same time that it furnishes a significant supply of elemental calcium by weight of the salt than other salts and has greater bioavailability.

Optionally the calcium salt can be pregranulated with maltodextrins or with pregelatinized starch, in order to obtain a raw material with adequate flow and compressibility characteristics.

Mixtures of calcium carbonate/maltodextrin with a weight ratio of 95/5 and 90/10 are preferably used.

Optionally the vitamin D can be stabilized with antioxidants.

Preferably vitamin D stabilized with DL-α-tocopherol was used.

The fluorine is supplied in the form of a salt.

Preferably the fluorine salt is chosen among sodium monofluorophosphate and sodium fluoride.

In a more preferred variant, the fluorine salt is sodium monofluorophosphate in an amount between 15 and 230 mg, more preferably between 50 and 200 mg.

The disintegrant is preferably chosen among crospovidon, sodium croscarmelose, guar gum, sodium glycolate of starch and cellulose derivatives, like hydroxypropylcellulose with a low degree of substitution.

In a more preferred variant the disintegrant is chosen among crospovidon, sodium croscarmelose, sodium glycolate of starch and hydroxypropylcellulose with a low degree of substitution and is incorporated in an amount between 1 and 10 wt % of the formulation.

In an even more preferred variant the disintegrant is hydroxypropylcellulose with a low degree of substitution (L-HPC), added in an amount between 4 and 6 wt % of the formulation.

Preferably L-HPC is chosen among the varieties (LH-11 and LH-21) with different particle size, density and degree of substitution.

The sweetener is preferably a sweetener of intense flavor chosen among the group formed by aspartame, sodium saccharine, sodium cyclamate, potassium acesulfame and their mixtures and is added in an amount between 0.1 and 1% by weight of the formulation.

In a more preferred variant the sweetener of intense flavor is selected among aspartame and mixtures containing it.

In an even more preferred variant the sweetener of intense flavor is aspartame added in an amount between 0.15 and 0.55% by weight of the formulation.

The sweetener of intense flavor can be optionally accompanied by at least one flavoring chosen from the group formed by oils of orange, lemon, strawberry, forest fruits, mint and anise in an amount between 0.01 and 1% by weight of the formulation.

The orally dispersible tablets of the present invention can also contain other pharmaceutically acceptable excipients.

In the preferred form an effervescent agent is present consisting of an agent capable of liberating CO₂ combined with an agent that induces liberation of CO₂.

The agent capable of liberating CO₂ can be selected among carbonates and bicarbonates of alkali metals.

In a particularly preferred variant the agent capable of liberating CO₂ is calcium carbonate.

The agent that induces liberation of CO₂ can be chosen among organic acids, their acid salts and their mixtures.

In a more preferred variant the agent that induces liberation of CO₂ is an organic acid selected among tartaric acid, malic acid, fumaric acid, succinic acid, ascorbic acid, maleic acid, citric acid, acid salts of said acids and their mixtures.

In an even more preferred variant the organic acid is chosen among anhydrous citric acid, tartaric, ascorbic acids and their mixtures. And more preferably it is anhydrous citric acid in an amount between 2 and 25 wt % of the formulation.

The agent that induces CO₂ liberation is preferably in solid form and can be used in different particle sizes chosen between the powder and granular form.

In a particularly preferred variant, the effervescent agent consists of calcium carbonate/citric acid in a weight ratio between 10/1 and 2/1 but preferably between 8/1 and 4/1.

Preferably the tablets of the present invention will also contain at least one diluent-binder selected from the group formed by lactose, plasdon, maltodextrin, microcrystalline cellulose and dextrates in an amount between 1 and 10 wt % of the formulation.

In a particularly preferred variant the diluent is lactose incorporated in an amount between 1 and 5 wt % of the formulation.

Preferably the formulation will also contain at least one lubricant chosen among the group formed by stearic acid, magnesium stearate, sodium stearylfumarate, calcium stearate and polyethylene glycols in an amount between 0.5 and 5 wt % of the formulation.

In a particularly preferred variant the lubricant is stearic acid, added in an amount between 1 and 3 wt % of the formulation.

The tablets of the present invention can also contain at least one sweetener with a not very intense taste selected in the group formed by sorbitol, mannitol, xylitol, fructose, maltose, maltitol, lactitol and their mixtures in an amount between 1 and 20 wt % of the formulation.

Nevertheless, in the preferred variant the tablets will not contain a sweetener of mild flavor.

In a particularly preferred variant the orally dispersible tablets of the present invention contain:
between 1250 mg and 1750 mg calcium carbonate;
between 50 mg and 150 mg hydroxypropylcellulose with a low degree of substitution;
between 100 mg and 450 mg citric acid;
between 4 mg and 15 mg aspartame;
optionally between 300 IU and 500 IU vitamin D;
optionally between 50 mg and 200 mg monofluorophosphate.

Both the excipients and the active principles used in the tablets of the present invention are known and can be obtained from commercial sources.

The orally dispersible tablets of the present invention are useful in the treatment or prevention of osteoporosis and other diseases characterized by loss of bone mass.

### EXAMPLES

The invention is illustrated with the following nonlimiting examples:

### Example 1

### Preparation Processes

The tablets of the present invention are prepared following the direct compression technique.

In a particular variant the sweetener, disintegrant and optionally vitamin D are mixed.

The premix is situated in a mixer combined with the calcium salt and optionally organic acid, flavoring, diluent-binder and/or lubricant. The obtained mixture is compressed in a rotary compression machine.

In another particular variant the calcium salt, disintegrant and optionally organic acid and/or diluent-binder are premixed. The premix is withdrawn from the mixer and a quarter of the weight of it is introduced to the same mixer. The sweetener and optionally vitamin D and/or flavoring are incorporated in the form of a sandwich (in the interior) and mixed.

Another quarter of the premix is added and mixed. This procedure is repeated with each quarter part of the remaining premix. Lubricant is optionally added and mixed. Finally the resulting mixture is compressed in a rotary machine.

### Example 2

### Formulations

The obtained orally dispersible tablets can have adequate technical characteristics and were well accepted in tests with healthy volunteers.

In the following detailed formulations, calcium carbonate 95% MD and 90% MD denotes calcium carbonate granulated with 5% and 10% by weight maltodextrin respectively; cholecalciferol represents vitamin D3 stabilized with DL-α-tocopherol in which 1 mg of cholecalciferol is equivalent to 100 IU active principle.

The tests to determine tensile strength, friability and disintegration time of the tablets were carried out according to the Royal Spanish Pharmacopoeia, 2^{nd} edition, 2002.

### Formula A

Calcium carbonate 95% MD [see original for amounts]
Granular citric acid
Lactose monohydrate
Sodium croscarmelose
Magnesium stearate

### Aspartame

### Orange oil

Tensile strength of the tablet (kgf)=7.4

### Friability (%)=3.54

Disintegration time (min)=1.8

### Formula B

### Cholecalciferol

Calcium carbonate 90% MD
Anhydrous granular citric acid

### L-HPC LH11

Lactose monohydrate
Stearic acid

### Aspartame

### Orange oil

Tensile strength of the tablet (kgf)=8.7

### Friability (%)=0.28

Disintegration time (min)=0.7

### Formula C

### Cholecalciferol

Calcium carbonate 95% MD
Granular citric acid
Sodium croscarmelose
Lactose monohydrate
Magnesium stearate

### Aspartame

### Lemon oil

Tensile strength of the tablet (kgf)=5.5
Disintegration time (min)=1.0-1.5

### Formula D

### Cholecalciferol

Calcium carbonate 95% MD
Granular citric acid

### Crospovidon

Lactose monohydrate
Stearic acid

### Aspartame

### Orange oil

Tensile strength of the tablet (kgf)=7.3

### Friability (%)=2.23

Disintegration time (min)=1.0-1.5

### Formula E

### Cholecalciferol

Calcium carbonate 95% MD
Granular citric acid
Lactose monohydrate
Sodium starch glycolate
Magnesium stearate

### Aspartame

### Orange oil

Tensile strength of the tablet (kgf)=6.2
Disintegration time (min)=1.0-1.5

### Formula F

### Cholecalciferol

Calcium carbonate 95% MD
Granular citric acid

### Mannitol

### L-BPC LH-21

Lactose monohydrate
Magnesium stearate

### Aspartame

### Strawberry oil

Tensile strength of the tablet (kgf)=8.7
Disintegration time (min)=1.0-1.8

### Formula G

### Cholecalciferol

Calcium carbonate 95% MD
Granular citric acid

### L-HPC LH-21

Lactose monohydrate
Stearic acid

### Aspartame

### Orange oil

Tensile strength of the tablet (kgf)=8.7

### Friability (%)=2.75

Disintegration time (min)=1.4

### Formula H

### Cholecalciferol

Calcium carbonate 95% MD
Anhydrous granular citric acid

### Guar gum

Lactose monohydrate
Magnesium stearate

### Aspartame

### Orange oil

Tensile strength of the tablet (kgf)=5.8
Disintegration time (min)=1.0-1.5

### Formula I

### Cholecalciferol

Calcium carbonate 95% MD
Anhydrous granular citric acid

### L-HPC LH-21

Sodium croscarmelose
Lactose monohydrate
Stearic acid

### Aspartame

### Orange oil

Tensile strength of the tablet (kgf)=7.0
Disintegration time (min)=1.5-2.0

### Formula J

### Cholecalciferol

Calcium carbonate 95% MD
Granular citric acid
Granular tartaric acid

### L-HPC LH-21

Lactose monohydrate
Stearic acid

### Aspartame

### Orange oil

Tensile strength of the tablet (kgf)=8.0

### Friability (%)=1.64

Disintegration time (min)=0.9

### Formula K

### Cholecalciferol

Calcium carbonate 95% MD
Anhydrous granular citric acid

### L-HPC LH-11

Lactose monohydrate
Stearic acid

### Aspartame

Sodium saccharine

### Orange oil

Tensile strength of the tablet (kgf)=8.0

### Friability (%)=2.61

Disintegration time (min)=1.3

### Formula L

### Cholecalciferol

Calcium carbonate 95% MD
Anhydrous granular citric acid
Granular ascorbic acid

### L-HPC LH-11

Lactose monohydrate
Stearic acid

### Aspartame

### Orange oil

Tensile strength of the tablet (kgf)=8.7

### Friability (%)=2.51

Disintegration time (min)=1.7

### Formula M

### Cholecalciferol

Calcium carbonate 90% MD
Sodium monofluorophosphate
Citric acid

### L-HPC LH-11

Magnesium stearate

### Aspartame

Oil of forest fruits

What is claimed is:

1. An orally dispersible pharmaceutical composition, comprising:
between 15 and 35 wt % of the composition of elemental calcium;
at least one disintegrant; and
at least one sweetener.

2. The composition according to claim 1 wherein said composition contains between 400 mg and 700 mg of elemental calcium.

3. The composition according to claim 1 further comprising between 100 IU and 500 IU of vitamin D.

4. The composition according to claim 1 further comprising 2 mg and 30 mg of fluorine.

5. The composition according to claim 1 wherein said elemental calcium is derived from a calcium salt selected from the group consisting of calcium carbonate, calcium pidolate, calcium lactate, calcium citrate, calcium gluconate, calcium chloride, calcium glucoheptonate, calcium phosphate, calcium glycerophosphate, and mixtures thereof.

6. The composition according to claim 1 wherein said elemental calcium is in the form of a salt selected from the group consisting of calcium carbonate, calcium gluconate, calcium lactate, calcium citrate, and mixtures thereof.

7. The composition according to claim 1 wherein said elemental calcium comprises calcium carbonate in an amount between 45 and 90 wt % of the composition.

8. The composition according to claim 7, wherein said calcium carbonate is present in an amount between 1000 and 1750 mg.

9. The composition according to claim 1, wherein said elemental calcium is in the form of a calcium salt that is granulated with a binder selected from the group consisting of maltodextrins and pregelatinized starch.

10. The composition according to claim 1, wherein the disintegrant is selected from the group consisting of crospovidon, sodium starch glycolate, sodium croscarmelose and hydroxypropylcellulose with a low degree of substitution, in an amount between 1 and 10 wt % of the composition.

11. The composition according to claim 10, wherein the disintegrant is hydroxypropylcellulose with a low degree of substitution in an amount between 4 and 6 wt % of the composition.

12. The composition according to claim 1, wherein the sweetener is a sweetener with an intense flavor selected from the group consisting of aspartame, sodium saccharine, sodium cyclamate, potassium acesulfame, and mixtures thereof, in an amount between 0.1 and 1% of the composition.

13. The composition according to claim 12, wherein said sweetener comprises aspartame in an amount between 0.15 and 0.055 wt % of the composition.

14. The composition according to claim 1, further comprising one or more flavoring selected from the group consisting of orange, lemon, strawberry essential oils, forest fruits, mint and anise, in an amount between 0.01 and 1 wt % of the composition.

15. The composition according to claim 1, further comprising an effervescent agent consisting of at least one agent to liberate CO₂ selected from the group consisting of alkali metal carbonates, alkali metal bicarbonates and mixtures thereof, combined with at least one agent that induces liberation of CO₂ selected from the group consisting of organic acids, acid salts of organic acids, and their mixtures.

16. The composition according to claim 15, wherein the agent that liberates CO₂ is calcium carbonate.

17. The composition according to claim 15, wherein the agent that induces liberation of CO₂ is selected from a group consisting of tartaric acid, malic acid, fumaric acid, succinic acid, ascorbic acid, maleic acid, citric acid, salts of said acids, and mixtures thereof.

18. The composition according to claim 15, wherein the at least one agent comprises anhydrous citric acid in an amount between 2 and 25 wt % of the composition.

19. The composition according to claim 1, further comprising a diluent-binder selected from the group consisting of lactose, plasdon, maltodextrin, microcrystalline cellulose, dextrates, and mixtures thereof, in an amount between 1 and 10 wt % of the composition.

20. The composition according to claim 1, further comprising a lubricant selected from the group consisting of stearic acid, magnesium stearate, sodium stearyl fumarate, calcium stearate, polyethylene glycols, and mixtures thereof, in an amount between 0.5 and 5 wt % of the composition.

21. The composition according to claim 1, comprising,
between 1250 and 1750 mg calcium carbonate;
between 50 mg and 150 mg hydroxypropylcellulose with a low degree of substitution;
between 100 mg and 450 mg citric acid; and
between 4 mg and 15 mg aspartame.

22. The composition according to claim 21, further comprising between 300 IU and 500 IU of vitamin D.

23. The composition according to claim 21 further comprising between 50 mg and 200 mg of sodium monofluorophosphate.

24. A method for treating diseases characterized by loss of bone mass, comprising administering to a patient an effective amount of the composition as set forth in claim 1.

25. A method for preparing a pharmaceutical composition, comprising;
premixing a sweetener and a disintegrant, to form a premix;
mixing the premix with a calcium salt and one or more of an organic acid, flavoring, diluent-binder and a lubricant, to form a mixture; and
compressing the mixture.

26. A method for preparing a pharmaceutical composition, comprising:
(a) premixing a calcium salt and a disintegrant to form a premix;
(b) withdrawing the premix from a mixer and introducing to the mixer a first portion of the premix;
(c) incorporating in sandwich form a sweetener to the premix;
(d) mixing said sweetener with said premix;
(e) adding a second portion of the premix and mixing;
f. repeating steps c) and d) using a third portion of the premix; and
g. compressing a resulting mixture.

27. The method of claim 26, further comprising adding at least one of vitamin D, an organic acid and a diluent-binder during said premixing step.

28. The method of claim 26, further comprising adding a lubricant in one of steps (a-f).
